(19) United States
(12) Patent Application Publication
(10) Pub. No.: US 2002/0111305 A1
(43) Pub. Date: Aug. 15, 2002

(54) Antiviral peptides

(76) Inventors: van Nieuw Amerongen, Breukelen (NL); Engelmundus Cornelis Ignatius Veerman, Volendam (NL); Willem van 't Hof, Leiden (NL); Peter Hendricus Nibbering, Voorhout (NL)

Correspondence Address: FOLEY, HOAG & ELIOT, LLP, PATENT GROUP, ONE POST OFFICE SQUARE, BOSTON, MA, 02109, US

(21) Appl. No.: 09/872,864

(22) Filed: Jun. 1, 2001

Related U.S. Application Data
(63) Continuation of application No. PCT/NL99/00732, filed on Dec. 1, 1999.

(30) Foreign Application Priority Data
Dec. 1, 1998 (NL) 1010692

(51) Int. Cl. A61K 38/16; A61K 38/10; C07K 14/00

(52) U.S. Cl. 514/12; 514/13; 514/14; 514/15; 530/325; 530/324; 530/326

(57) ABSTRACT

The invention relates to peptides for use as antiviral agent, consisting of an amino acid chain which contains a domain of 10 to 25 amino acids, wherein the majority of the amino acids of the one half of the domain are positively charged amino acids and the majority of the amino acids of the other half of the domain are uncharged amino acids. The invention further relates to oligomers of these peptides consisting of at least two such peptides which are coupled to each other, optionally via a spacer, for use as antiviral agent, in addition to the use of the peptides and/or oligomers for the manufacture of a medicine for treating viral infections.

The present invention relates to new peptides, derived from natural saliva peptides, with an antiviral activity.

Viruses can be combatted only with great difficulty using chemotherapeutic agents. One reason for this is the fact that the growth of viruses is closely linked to cell functions of the host. When viruses are combatted the host cell will also be subject to at least some irrevocable damage. In addition, the classical antibiotics used to combat bacteria and other micro-organisms have no or hardly any effect on viruses. Vaccinations are effective in respect of a number of virus infections, while antiviral therapy is available in only a few virus infections (Herpes Simplex Virus (HSV) and Human Immune-deficiency Virus (HIV)). The antiviral agents in question are virus-specific. No effective antiviral agent is known in most virus infections.

The object of the present invention is to provide new antiviral agents with wide activity, i.e. with an activity in respect of a plurality of viruses, both DNA and RNA viruses, irrespective of whether they possess a virus envelope or not.

This is achieved with the invention by using as antiviral agent peptides which consist of an amino acid chain containing a domain of 10 to 25 amino acids, wherein the majority of the amino acids of the one half of the domain are positively charged amino acids and the majority of the other half of the domain are uncharged amino acids.

The structure of these peptides has a number of variations. Firstly, the active domain can form an α-helix, of which at least a majority of the positions 1, 2, 5, 6, 9 (12, 13, 16, 19, 20, 23 and 24) contains a positively charged amino acid, position 8 is a positively or an uncharged amino acid and at least a majority of the positions 3, 4, 7, 10, (11, 14, 15, 17, 18, 21, 22, 25) contains an uncharged amino acid. These peptides have a lateral amphipathicity, i.e. a maximum hydrophobic moment at 1000. Stated simply, these peptides are hydrophobic on the left side and hydrophilic on the right side or vice versa. These peptides are referred to herein as “type I”.

The domain can further form an a-helix, of which at least a majority of the positions 1, 2, 5, 6, 9 (12, 13, 16, 19, 20, 23 and 24) contains an uncharged amino acid, position 8 is a positive or an uncharged amino acid and at least a majority of the positions 3, 4, 7, 10, (11, 14, 15, 17, 18, 21, 22, 25) contains a positively charged amino acid. These peptides have a lateral amphipathicity, i.e. a maximum hydrophobic moment at 1000. Stated simply, these peptides are hydrophobic on the right side and hydrophilic on the left side or vice versa. These peptides are designated “type II” herein and are in principle mirror-symmetrical to type I peptides.

In addition, the domain can form an α-helix, wherein at least a majority of the positions 1 to 6 (or 7 or 8 or 9 or 10 or 11 or 12) contains an uncharged amino acid and a positively charged amino acid is found at position 7 (or 8 or 9 or 10 or 11 or 12 or 13) to 25. These peptides have a longitudinal amphipathicity, i.e. a minimum hydrophobic moment at 1000. These peptides are hydrophobic on their “top” and hydrophilic on their “bottom”. Such peptides are designated “type III”.

Conversely, the domain can form an a-helix, wherein at least a majority of the positions 1 to 6 (or 7 or 8 or 9 or 10 or 11 or 12) contains a positively charged amino acid and an uncharged amino acid is found at position 7 (or 8 or 9 or 10 or 11 or 12 or 13) to 25. These peptides likewise have a longitudinal amphipathicity and therefore a minimum hydrophobic moment at 1000. These peptides are hydrophobic on their “bottom” and hydrophilic on their “top”. Such peptides are designated “type IV”.

Finally, the domain can form a so-called β-strand and contain a positively charged amino acid on at least a majority of the positions 1, 3, 5, 7, 9 (11, 13, 15, 17, 19, 21, 23 and 25) and an uncharged amino acid on at least a majority of the positions 2, 4, 6, 8, 10, (12, 14, 16, 18, 20, 22, 24). Such a β-strand is laterally amphipathic and has a maximum hydrophobic moment at 180°. The β-strand structure is flatter than the α-helix and, stated simply, is hydrophobic on the left and hydrophilic on the right or vice versa. These are “type V” peptides.

The positively charged amino acids are preferably chosen from the group consisting of ornithine (O), lysine (K), arginine (R) and histidine (H), while the uncharged amino acids are preferably chosen from the group consisting of the aliphatic amino acids glycine (G), alanine (A), valine (V), leucine (L), isoleucine (I), the amino acids with a dipolar side chain methionine (M), asparagine (N), glutamine (Q), serine (S), threonine (T), the amino acids with an aromatic side chain phenylalanine (F), tyrosine (Y), tryptophan (W) Amino acids on the border between hydrophilic and hydrophobic can be chosen from both groups or from the remaining amino acids.

Hardly any difference in activity can in principle be detected when one of the positive amino acids and/or one of the uncharged amino acids is replaced by a random amino acid. The majority of the positively charged amino acids is therefore preferably the total number of positively charged amino acids minus 1 and the majority of the uncharged amino acids is preferably the total number of uncharged amino acids minus 1.

The domain can be a part of a larger peptide but can itself also make up the entire peptide. When the domain forms part of a larger peptide, the C-terminal and/or N-terminal amino acids which are then additionally present can be random amino acids.

In addition, these domains can also form part of more complex structures, such as oligomeric peptides, hybrid peptides (together with another peptide, lipids, oligosaccharides, (radioactive) labels, organic receptor ligands etc.) and peptide conjugates. The peptide agent can be enclosed in for instance liposomes of virions so as to better ensure the intercellular activity of the peptide.

The following peptides of the type I are particularly recommended:

| | |
|---|---|
| KRLFKELKFSLRKY | (peptide 3) |
| KRLFKELLFSLRKY | (peptide 4) |
| KRLFKELKKSLRKY | (peptide 5) |
| KRLFKELLKSLRKY | (peptide 6) |
| OOLFOELOOSLOOY | (peptide 7) |
| OOLFOELLOSLOOY | (peptide 8) |
| KRLFKKLKFSLRKY | (peptide 9) |
| KRLFKKLLFSLRKY | (peptide 10) |

A preferred peptide of the type II has the following amino acid sequence:

LLLFLLKKRKKRKY (peptide 11).

The peptides according to the invention can also contain further modifications. These modifications are for instance an N-terminal amide ring, for instance with acetic acid anhydride, or an alternative cleavage of the synthesis resin by which the C-terminus is modified. For this latter a replacement of the C-terminal carboxylic acid group by an amide, ester, ketone, aldehyde or alcohol group can be envisaged. Peptides with such a modification are for instance:

KRLFKELKFSLRKY-amide (peptide 12)

KRLFKELLFSLRKY-amide (peptide 13)

In addition to single peptides, oligomers can also be made. These are preferably linear oligomers of the peptides according to the invention. The coupling can be head-to-head and tail-to-tail as well as head-to-tail, either by direct synthesis or by post-synthetic enzymatic coupling. The advantage of oligomers of the peptides lies in a better efficacy and a wider spectrum of activity, as is illustrated in the examples. A spacer must usually be inserted. In direct synthesis of head-to-tail coupled oligomers a spacer can be inserted to size by the use of a chain of unnatural amino acids of the correct length, for instance β-alanine, γ-amino butyric acid, ε-amino caproic acid, etc. Hetero-difunctional coupling reagents, such as are commercially available for coupling peptide antigens to carrier proteins (for instance 1-ethyl-3-[3-dimethyl aminopropyl]carbodiimide (EDC), m-maleimidobenzoyl)-N-hydroxysuccinimide ester (MBS), N-succinimidyl 3-[pyridyldithio]propionate (SPDD) etc.) are used to make linear oligomers with an inserted spacer. For head-to-head and tail-to-tail couplings can be used trivalent amino acids such as asparagine acid (D), glutamine acid (E), ornithine (O), lysine (K), serine (S), cysteine.

Very suitable oligomers for use in the invention are the oligomers of peptides 10 and 11, with the following amino acid sequence:

α,ε-(KRLFKKLLFSLRKY)₂-K-amide (peptide 10-dimer)

α,ε-(LLLFLLKKRKKRKY)₂-K-amide (peptide 11-dimer)

The peptides described herein have no or hardly any haemolytic activity.

In vitro assays have demonstrated that the peptides described herein have no toxic effects in respect of human red blood cells and monkey kidney cells (vero-cells).

The peptides and/or oligomers thereof can be used according to the invention in or as an antiviral agent. Their antiviral activity will be further illustrated in the accompanying examples.

Also part of the invention is the use of the peptides and/or oligomers thereof for the manufacture of a medicine for the treatment of virus infections.

The peptides and constructs derived therefrom according to the invention can be used in different pharmaceutical forms of administration for the treatment of diverse viral disorders. Examples hereof are the development of (mouth) sprays, ointments, gels and lozenges for treating cold sores, aphthous ulcers and viral bronchial infections.

The peptides and oligomers according to the invention can be used in different pharmaceutical forms of administration for the treatment of cold sores, aphthous ulcers and viral bronchial infections. Particularly recommended are (mouth) spray, ointment, gel and lozenges.

The invention is further illustrated in the accompanying examples, which are only given by way of illustration and not to limit the invention in any way whatever.

### EXAMPLES

### Example 1

Peptide Synthesis

Peptides according to the invention were chemically synthesized as described by Van 't Hof et al. (1991) and Helmerhorst et al. (1997). Peptides were synthesized using the T-bag method, which was adapted for 9-fluorenylmethoxycarbonyl ((Fmoc) chemistry). p-Benzyloxybenzyl alcohol resins to which the first N-Fmoc-protected amino acids were already coupled, were arranged in the T-bags. The coupling reactions were performed in N,N-dimethyl formamide. After completion of the amino acid chain it was cleaved from the resin and the side chain protection groups were simultaneously removed with a mixture of 5% thioanisole, 5% phenol, 5% water and 85% trifluoroacetic acid. Purity analyses were performed by reversed-phase HPLC and showed one single peak with only few peptide contaminants (less than 5%).

All peptides were dissolved in 10 mM sodium phosphate buffer (NaPB), pH 7.4, to a concentration of 2 mg/ml and stored at −20° C. The exact peptide concentrations which were used in the antiviral assays were determined by amino acid analysis.

Table 1 gives an overview of the peptides 2 to 13 which were made in this manner. Peptides 1 and 2 of this table show respectively the histatin 5 and the C-terminal part thereof.

TABLE 1

| Peptide | Sequence |
|---|---|
| 1 | DSHAKRHHGYKRKFHEKHHSHRGY |
| 2 | KRKFHEKHHSHRGY |
| 3 | KRLFKELKFSLRKY |
| 4 | KRLFKELLFSLRKY |
| 5 | KRLFKELKKSLRKY |
| 6 | KRLFKELLKSLRKY |
| 7 | OOLFOELOOSLOOY |
| 8 | OOLFOELLOSLOOY |
| 9 | KRLFKKLKFSLRKY |
| 10 | KRLFKKLLFSLRKY |
| 11 | LLLFLLKKRKKRKY |
| 12 | KRLFKELKFSLRKY-amide |
| 13 | KRLFKELLFSLRKY-amide |
| 14 | KRKFHEKHHSHRGYC-CYGRHSHHKEHFKRK |
| 15 | YGRHSHHKEHFKRKC-CKRKFHEKHHSHRGY |
| 16 | ^{α}N, ^{ε}N-(KRKFHEKHHSHRGY)₂K-amide |
| 17 | ^{α}N, ^{ε}N-(KRLFKELKFSLRKY)₂K-amide |
| 18 | ^{α}N, ^{ε}N-(KRLFKKLKFSLRKY)₂K-amide |

### Example 2

Antiviral Activity Against Herpes Simplex Virus (HSV

In test phials 10 μl HSV (Department of Virology Academisch Ziekenhuis Leiden (Leiden University Hospital), lab strain 96-6700 P (TCID₅₀ 10⁵-10⁶), was supplemented with peptide and NaPB to 200 μl. For the positive control the peptide was replaced by a human neutrophil defensin pool (HNP₁₋₃). The test phials were subsequently incubated at 37° C. for 3 hours. Tenfold dilutions were then made in Dulbecco's Modified Eagles Medium (DMEM) with 2% foetal calf serum (FSCᵢ). Diverse peptides, synthesized in identical manner, were used as negative control in the experiments.

Vero-cells were isolated using detachment buffer (0.25% trypsin and 0.03% EDTA in PBS), washed and brought to a concentration of 2×10⁵ cells per ml DMEM +2% FCSᵢ. In 96 wells-plates (Nunclon) 100 μl cell suspension (2×10⁴ cells) was added per well. Acyclovir (ACV) was added as control to several wells about two hours (t=−2 hours) before infection. 50 μl of a dilution was added to each well at the point in time t=0.

After 3 days incubation in a CO₂-stove of 37° C. the cytopathological effect (cpe) was scored by counting under the microscope and the TCID₅₀ (tissue culture effective dose) was determined using the Reed & Muench method (Dulbecco & Ginsberg, “Virology”, JB Lippincott Co., Philadelphia, 2nd edition, 1988).

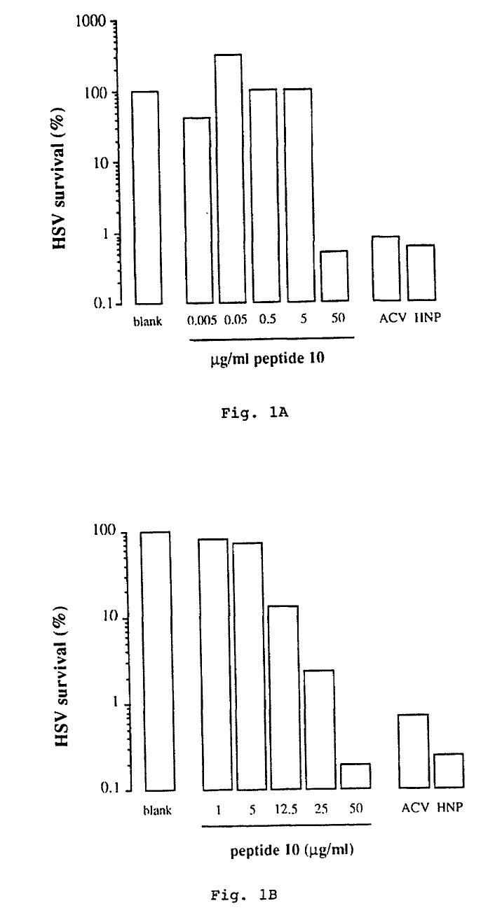

FIG. 1 shows that in sufficiently high concentrations (50 μg/ml) peptide 10 is at least as effective as acyclovir and HNP.

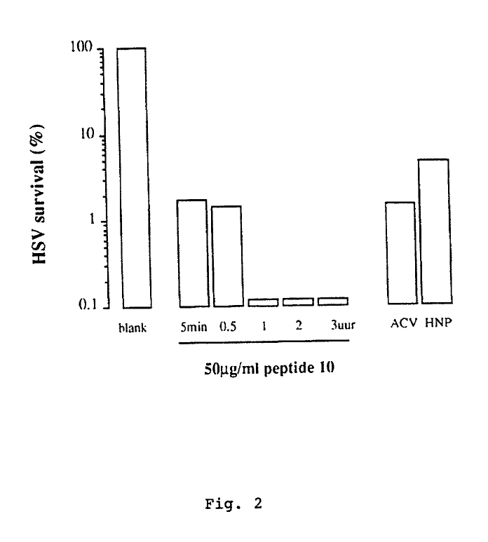

The effectiveness of peptide 10 in time was then determined. The concentrations of the controls amounted to 50 μg/ml for HNP₁₋₃ and to 5 μg/ml for acylclovir (ACV). The same test arrangement was used for this purpose as for the first part of the test and samples were taken after 5 and 30 minutes and after 1, 2 and 3 hours. The result is shown in FIG. 2.

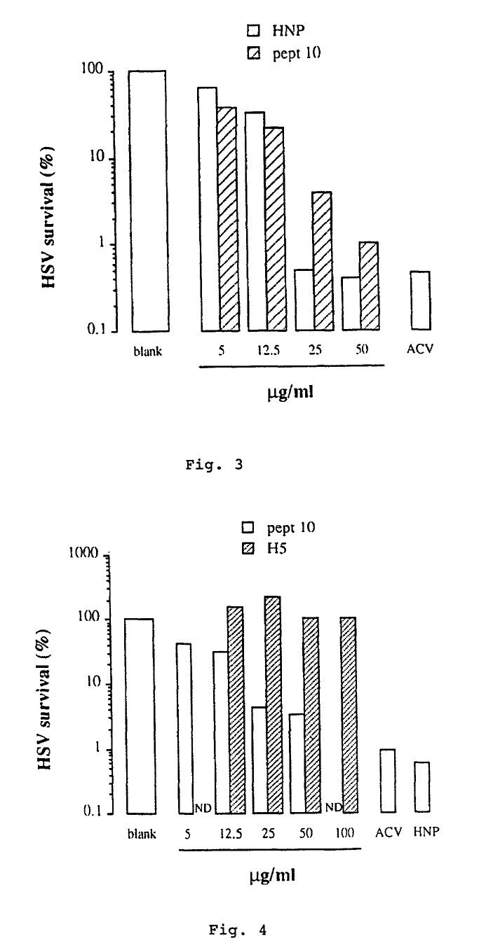

A comparison was then made in the same manner as described above between the activity of dilution series of HNP and peptide 10 according to the invention. Acyclovir was once again used as positive control. FIG. 3 shows that in high concentrations peptide 10 is more effective than HNP.

### EXAMPLE 3

Killing of the Measles Virus

Different concentrations of peptide 11 and a dimer of peptide 10 in PBS (pH 7.4) were mixed in test phials with 5 to 10 μl measles virus stock solution to a final volume of 200 Al and incubated at 37° C. for 3 hours. After the incubation period the test phials were placed on ice and immediately diluted serially in DMEM with 2% FCS, 100 U/ml penicillin G, 100 μg/ml streptomycin and 20 mM HEPES-buffer (pH 7.4). The serial dilutions were plated out on tissue culture mono-layers to determine the TCID₅₀ using the Reed & Münch method.

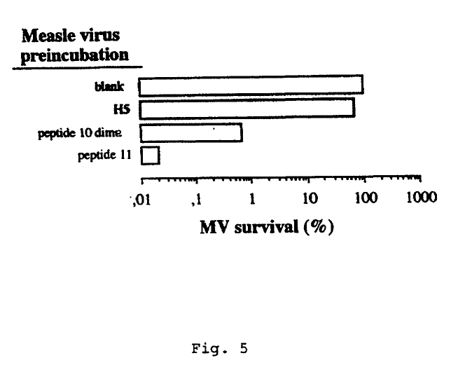

FIG. 5 shows the result. The different effects of the pre-incubation time on the TCID₅₀s of measles virus as a consequence of peptide 11 and of adenovirus as a consequence of the dimer of peptide 10 suggest that diverse activity mechanisms exist side by side.

### Example 4

Neutralization of Human Immuno-deficiency Virus Type 1 (HIV-1) by Peptides According to the Invention

A neutralization assay was performed on HIV-1 III-b as substantially described by Groenink M. et al., J. Virol 69, 523-527 (1995). In short, this assay comes down to the virus-neutralizing capacity of the different peptides being tested with an inoculum of 457 TCID₅₀HIV per ml per neutralization, which is incubated at 37° C. for two hours with a twofold dilution series of the peptide for testing (maximum 800 μg/ml final concentration). The incubations are performed in a 5 mM phosphate buffer. On day 7, 14 and 21 the neutralization is assessed on the basis of the detected cytopathic effect (syncytia-forming) on MT-2 cells. Table 2 gives the result of day 21.

TABLE 2
neutralizing capacity*

| peptide | μg/ml necessary for neutralization |
|---|---|
| his 5 | no neutralization |
| dh-5 | no neutralisation |
| peptide 4 | 200 |
| peptide 10 | 200 |
| peptide 11 | 25 |
| *concentration of peptide in μg/ml capable of neutralizing HIV-1 | |
| The table shows that peptide 11 has the strongest neutralizing activity. It was further found that only | |
| # peptide 11 was cytoxic at the highest concentration (800 μg/ml). | |

1. Peptides for use as antiviral agent, consisting of an amino acid chain which contains a domain of 10 to 25 amino acids, wherein the majority of the amino acids of the one half of the domain are positively charged amino acids and the majority of the amino acids of the other half of the domain are uncharged amino acids.

2. Peptides as claimed in claim 1, characterized in that the domain forms an α-helix and at least at a majority of the positions 1, 2, 5, 6, 9 (12, 13, 16, 19, 20, 23 and 24) contains a positively charged amino acid, at position 8 a positive or an uncharged amino acid and at least at a majority of the positions 3, 4, 7, 10, (11, 14, 15, 17, 18, 21, 22, 25) contains an uncharged amino acid.

3. Peptides as claimed in claim 1, characterized in that the domain forms an a-helix and at least at a majority of the positions 1 to 6 (or 7 or 8 or 9 or 10 or 11 or 12) contains an uncharged amino acid and at position 7 (or 8 or 9 or 10 or 11 or 12 or 13) to 25 a positively charged amino acid.

4. Peptides as claimed in claim 1, characterized in that the domain forms an α-helix and at least at a majority of the positions 1 to 6 (or 7 or 8 or 9 or 10 or 11 or 12) contains a positively charged amino acid and at position 7 (or 8 or 9 or 10 or 11 or 12 or 13) to 25 an uncharged amino acid.

5. Peptide as claimed in claim 1, characterized in that the domain forms a so-called β-strand and contains a positively charged amino acid on at least a majority of the positions 1, 3, 5, 7, 9 (11, 13, 15, 17, 19, 21, 23 and 25) and an uncharged amino acid on at least a majority of the positions 2, 4, 6, 8, 10, (12, 14, 16, 18, 20, 22, 24).

6. Peptides as claimed in claims 1-5, characterized in that the positively charged amino acids are chosen from the group consisting of ornithine (O), lysine (K), arginine (R) and histidine (H).

7. Peptides as claimed in claims 1-6, characterized in that the uncharged amino acids are chosen from the group consisting of the aliphatic amino acids glycine (G), alanine (A), valine (V), leucine (L), isoleucine (I), the amino acids with a dipolar side chain methionine (M), asparagine (N), glutamine (Q), serine (S), threonine (T), the amino acids with an aromatic side chain phenylalanine (F), tyrosine (Y), tryptophan (W).

8. Peptides as claimed in claims 1-7, characterized in that the majority of the positively charged amino acids is the total number of positively charged amino acids minus 1.

9. Peptides as claimed in claims 1-8, characterized in that the majority of the uncharged amino acids is the total number of uncharged amino acids minus 1.

10. Peptides as claimed in claims 1-9, characterized in that the domain makes up the entire peptide.

11. Peptides as claimed in claims 1-10, wherein the N-terminus is amidated.

12. Peptides as claimed in claims 1-11, wherein the C-terminal carboxylic acid group is replaced by an amide, ester, ketone, aldehyde or alcohol group.

13. Peptide as claimed in claim 2, of which the domain has the following amino acid sequence:
KRLFKELKFSLRKY (peptide 3).

14. Peptide as claimed in claim 2, of which the domain has the following amino acid sequence:
KRLFKELLFSLRKY (peptide 4).

15. Peptide as claimed in claim 2, of which the domain has the following amino acid sequence:
KRLFKELKKSLRKY (peptide 5).

16. Peptide as claimed in claim 2, of which the domain has the following amino acid sequence:
KRLFKELLKSLRKY (peptide 6).

17. Peptide as claimed in claim 2, of which the domain has the following amino acid sequence:
OOLFOELOOSLOOY (peptide 7).

18. Peptide as claimed in claim 2, of which the domain has the following amino acid sequence:
OOLFOELLOSLOOY (peptide 8).

19. Peptide as claimed in claim 2, of which the domain has the following amino acid sequence:
KRLFKKLKFSLRKY (peptide 9).

20. Peptide as claimed in claim 2, of which the domain has the following amino acid sequence:
KRLFKKLLFSLRKY (peptide 10).

21. Peptide as claimed in claim 3, of which the domain has the following amino acid sequence:
LLLFLLKKRKKRKY (peptide 11).

22. Oligomers of the peptides as claimed in claims 1-21, consisting of at least two such peptides which are coupled to each other, optionally via a spacer, for use as antiviral agent.

23. oligomers as claimed in claim 22, characterized in that the coupling of the monomeric peptides is head-to-head, i.e. with the N-terminal ends directed toward each other.

24. Oligomers as claimed in claim 22, characterized in that the coupling of the monomeric peptides is tail-to-tail, i.e. with the C-terminal ends directed toward each other.

25. oligomers as claimed in claim 22, characterized in that the coupling of the monomeric peptides is head-to-tail or tail-to-head, i.e. with the C-terminal end of the one monomer on the N-terminal ends of the second monomer or vice versa.

26. oligomer as claimed in claim 24, with the amino acid sequence α,ε-(KRLFKKLLFSLKY)₂-K-amide (peptide 10-dimer).

27. Oligomer as claimed in claim 24 with the amino acid sequence α,ε-(LLLFLLKKRKKRKY)₂-K-amide (peptide 11-dimer).

28. Use of peptides as claimed in claims 1-21 and/or oligomers as claimed in claims 22-25 for the manufacture of a medicine for treating viral infections.

29. Pharmaceutical composition for treating viral infections, comprising one or more peptides as claimed in claims 1-21 and/or oligomers as claimed in claims 22-25 and one or more suitable excipients.

30. Pharmaceutical composition as claimed in claim 29 in the form of a spray, ointment, gel or lozenge.

31. Constructs, wherein the peptides as claimed in claims 1-21 form part of hybrid peptides (together with another peptide, lipids, oligosaccharides, (radioactive) labels, organic receptor ligands etc.) and peptide polymer conjugates.


SEQUENCE LISTING (20 sequences)

SEQ ID NO: 1 - protein, 24 aa, Artificial Sequence
  Description of Artificial Sequence Synthetic peptide
DSHAKRHHGYKRKFHEKHHSHRGY

SEQ ID NO: 2 - protein, 14 aa, Artificial Sequence
  Description of Artificial Sequence Synthetic peptide
KRKFHEKHHSHRGY

SEQ ID NO: 3 - protein, 14 aa, Artificial Sequence
  Description of Artificial Sequence Synthetic peptide
KRLFKELKFSLRKY

SEQ ID NO: 4 - protein, 14 aa, Artificial Sequence
  Description of Artificial Sequence Synthetic peptide
KRLFKELLFSLRKY

SEQ ID NO: 5 - protein, 14 aa, Artificial Sequence
  Description of Artificial Sequence Synthetic peptide
KRLFKELLFSLRKY

SEQ ID NO: 6 - protein, 14 aa, Artificial Sequence
  Description of Artificial Sequence Synthetic peptide
KRLFKELLFSLRKY

SEQ ID NO: 7 - protein, 14 aa, Artificial Sequence
  Description of Artificial Sequence Synthetic peptide
XXLFXELXXSLXXY

SEQ ID NO: 8 - protein, 14 aa, Artificial Sequence
  Description of Artificial Sequence Synthetic peptide
XXLFXELLXSLXXY

SEQ ID NO: 9 - protein, 14 aa, Artificial Sequence
  Description of Artificial Sequence Synthetic peptide
KRLFKKLKFSLRKY

SEQ ID NO: 10 - protein, 14 aa, Artificial Sequence
  Description of Artificial Sequence Synthetic peptide
KRLFKKLLFSLRKY

SEQ ID NO: 11 - protein, 14 aa, Artificial Sequence
  Description of Artificial Sequence Synthetic peptide
LLLFLLKKRKKRKY

SEQ ID NO: 12 - protein, 14 aa, Artificial Sequence
  Description of Artificial Sequence Synthetic peptide
KRLFKELKFSLRKY

SEQ ID NO: 13 - protein, 14 aa, Artificial Sequence
  Description of Artificial Sequence Synthetic peptide
KRLFKELLFSLRKY

SEQ ID NO: 14 - protein, 30 aa, Artificial Sequence
  Description of Artificial Sequence Synthetic peptide
KRKFHEKHHSHRGYCCYGRHSHHKEHFKRK

SEQ ID NO: 15 - protein, 30 aa, Artificial Sequence
  Description of Artificial Sequence Synthetic peptide
YGRHSHHKEHFKRKCCKRKFHEKHHSHRGY

SEQ ID NO: 16 - protein, 29 aa, Artificial Sequence
  Description of Artificial Sequence Synthetic peptide
KRKFHEKHHSHRGYKRKFHEKHHSHRGYK

SEQ ID NO: 17 - protein, 29 aa, Artificial Sequence
  Description of Artificial Sequence Synthetic peptide
KRLFKELKFSLRKYKRLFKELKFSLRKYK

SEQ ID NO: 18 - protein, 29 aa, Artificial Sequence
  MOD_RES (29): AMIDATION
KRLFKKLKFSLRKYKRLFKKLKFSLRKYK

SEQ ID NO: 19 - protein, 29 aa, Artificial Sequence
  Description of Artificial Sequence Synthetic peptide
KRLFKKLLFSLRKYKRLFKKLLFSLRKYK

SEQ ID NO: 20 - protein, 29 aa, Artificial Sequence
  Description of Artificial Sequence Synthetic peptide
LLLFLLKKRKKRKYLLLFLLKKRKKRKYK